(19) United States
(12) Patent Application Publication
(10) Pub. No.: US 2004/0053218 A1
(43) Pub. Date: Mar. 18, 2004

(54) Functional proteomics using double phage display screening

(76) Inventors: Robert Stewart Gilmour, Little Abington Cambridge (GB); Keith Wright Marvin, Auckland (NZ)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/433,738

(22) PCT Filed: Dec. 7, 2001
(86) PCT No.: PCT/US01/47836

(51) Int. Cl. C12Q 1/70

(52) U.S. Cl. 435/5

(57) ABSTRACT

A use of identifying a protein by double phage screening.

### FIELD OF THE INVENTION

The present invention relates generally to the field of proteomics and, more specifically, to protein identification. The present invention exploits phagemid display to give a high-sensitivity, high through-put screen for structure/functional relationships of proteins identified by proteome analysis.

### BACKGROUND OF THE INVENTION

It has been known for a long time that the growth of tumors depends not only on the rate of cell proliferation, but also on the rate of cell death. An increase in cell proliferation and a decrease in apoptosis cumulatively results in an increase in cell numbers, which is the most relevant characteristic of tumors. From this perspective insulin-like growth factor-I (IGF-I) plays a very important role in tumor growth, because it stimulates cell proliferation, confers anchorage-independence and protects cells from cell death in general, and apoptosis in particular. Additionally, IGF-I also has important effects on glucose uptake and protein synthesis.

The biological actions of IGF-I are mediated through activation of the IGF-I receptor which triggers autophosphorylation of the receptor and also phosphorylation of the insulin receptor substrate-I (IRS-I). Tyrosyl-phosphorylated receptor and IRS-I interact with numerous SH₂ domain containing proteins which in turn activate both the Raf-MAPKK-MAPK cascade and the phosphatidylinositol-3 kinase/protein kinase B (PI3K/PKB) pathway. The detailed mechanisms by which these two pathways transmit the IGF-I dependent signals from cytosol to the nucleus and eventually control the cell cycle are still poorly understood. The traditional concept has been that the cytoplasmic pool of activated protein kinases phosphorylate transcription factors in the cytoplasm, which are consequently translocated to the nucleus. However, this explanation is in apparent contradiction with the fact that MAP kinase itself translocates to the nucleus immediately following the stimulation of IGF-I and other growth factors. Moreover, a recent study has shown that the nuclear translocation of MAPK and the subsequent phosphorylation of nuclear targets are crucial for the growth factor-induced gene expression and cell proliferation.

Previous work suggested that the generation of cytoplasmic signals is only part of the picture and that their entry into the nucleus and their subsequent action therein are a critical determinant of the phenotypic response. Thus, in the case of IGF-I it was demonstrated that cell cycle progression is dependent on the activation of an entirely new cycle. The biochemical mechanisms involved in this pathway are wholly analogous to those known to occur for plasma membrane receptor signal transduction except that in the case of the IGF-I response, the second messenger, diacylglycerol (DAG), is produced downstream of the cytoplasmic signaling cascade and is only found in the nucleus. Our research has shown that DAG results from the hydrolysis of nuclear PI lipids by a nuclear phospholipase C which is stimulated by the translocation of active MAP kinase into the nucleus. The accumulation of nuclear DAG in turn activates protein kinase C (PKC) which simultaneously translocates to the nucleus by an unknown mechanism and phosphorylates a number of proteins involved in cell cycle progression through the G1/S and G2/M checkpoints.

Thus the notion that the cytoplasm generates a tapering hierarchy of signals that terminate at the nucleus as a refined, unambiguous output no longer appears to be the case. On the contrary, the nucleus retains a good deal of signaling complexity and, in the case of the IGF-I, further expands it through additional intranuclear mechanisms. The cell response must ultimately depend on a resolution of these signals through phosphorylation-dependent changes in the properties of downstream, effector protein targets within the nucleus. In considering the role of the nuclear PI cycle in this process it is important to put its known effects into temporal context. Activation of nuclear PLC occurs within 1 minute of IGF-I stimulation, reaching a maximum at 15 minutes and returning to baseline after 30 minutes. DAG production and PKC activation lag behind these events by 10-15 minutes. However, the first effect on the cell cycle per se is not seen until 1-2 hours after IGF-I stimulation. Clearly, there is a crucial gap in our knowledge of what occurs in the period immediately following the activation of the nuclear PI cycle. We hypothesize the existence of a number of key post-cycle events 15-60 minutes after IGF-I stimulation which prepare the nucleus for cell division. To date there is no clear idea of these mechanisms or the nuclear effector proteins involved. Possible candidates include transcription factors (such as Elk1/TCF) which regulate gene expression, structural proteins (such as histone H1 and H2A, and lamin B) involved in nuclear architecture or enzymes (such as topoisomerase I and II) which catalyze a variety of nuclear reactions. However, many of these results are based on in vitro studies and their physiological relevance is uncertain.

Therefore, we systematically sought to identify the nuclear protein targets of IGF-I action using the latest developments of comparative proteome analysis, to determine the signaling pathways that lead to their phosphorylation and to assess their relevance to the initiation of the cell cycle. The ultimate goal being to provide an understanding of the mechanisms by which these nuclear proteins direct the processes of cell proliferation and survival and to provide a basis for rational drug design for novel anti-tumor therapies.

The term “proteome” refers to the spectrum of proteins that make up the skeleton and working parts of the cell. In disease subtle changes occur in the proteome; a few proteins change in amount or sub-cellular location, for example. Knowing what these proteins are and do provides a basic understanding of the disease as well as information for designing new therapeutic drugs.

To obtain further information about a protein including sub-cellular location, turnover rate, post-translational modification, covalent and noncovalent associations, and how all this is affected by different external and internal conditions it is necessary to study the proteins themselves. Only then can subtle changes be appreciated, like tissue-dependent variable post-translational modification of the same protein (e.g., human serotransferrin in plasma and cerebrospinal fluid) or the processing of a single polypeptide to produce many different products (e.g., the post-translational cleavage of protachykinin beta precursor into three peptide hormone products).

The proteome of the cell can be separated out and displayed as a two-dimensional matrix of hundreds to thousands of individual protein spots that form a reproducible pattern. Disease or changes in cell function produce recognizable changes in this pattern. A problem has been to identify the protein because the amounts present in the average spot are often outside the range of conventional biochemical analysis. For proteomics to be biologically meaningful requires a prior knowledge of an identified protein's function or a means of readily determining its functional relevance in vivo.

A decade ago a group in Cambridge, now Cambridge Antibody Technology (CAT), isolated the repertoire of genes that code for all the antibodies in a human. They then isolated just those parts of the antibody genes that code for the recognition and binding of foreign proteins. They transferred these gene fragments into a bacterial virus in such a way that the antibody recognition protein fragments were displayed on the surface of the virus and could function much as they do in the parent antibody. Each virus displays a different member of the original antibody repertoire. Since then CAT have artificially introduced even more diversity into this repertoire to display the equivalent of a billion different antibodies. This out-performs the body's capacity to diversity its own antibody repertoire by a long shot and means that there now exists a specific phage antibody that recognizes virtually every organic molecule known to nature. However, these antibody phagemid displays were virtually useless in identifying unknown proteins as they contained a myriad of recognition binding sites. In other words, an unknown protein was being bound by an unknown antibody.

Thus, a link was needed in order to use the antibody phage displays to identify the proteins isolated on proteomic gels. The present invention provides such a link.

### SUMMARY OF THE INVENTION

The present invention is directed to a mehtod of identifying a protein by double phage screening.

In one aspect of the invention there is a method of identifying a first protein, or a second protein containing an idiotypic region of the first protein, from a tissue of interest by double phage screening, said method comprising:

(a) contacting said first protein with an antibody phagemid display library to form a complex between said first protein and at least one member of said library;

(b) screening a cDNA phagemid display library of the proteome of the tissue of interest with the complex-forming antibody phage to identify a protein-specific phagemid that displays a second protein that binds the antibody phagemid; and

(c) identifying the second protein from the cDNA of the protein-specific phagemid.

In a second aspect there is provided a method of identifying a first protein, or a second protein containing an idiotypic region of the first protein, from a tissue of interest by ribosome-phage screening, said method comprising:

(a) contacting said first protein with an antibody phagemid display library to form a complex between said protein and at least one member of said library;

(b) screening a ribosome display library of the proteome of the tissue of interest with the complex-forming antibody phage to identify a protein-specific ribosome that displays a second protein that binds the antibody phagemid; and

(c) identifying the second protein from the cDNA of the protein-specific ribosome.

In an embodiment when the antibody phage yield is low, the antibody phage DNA is amplified prior to re-infecting bacteria.

### DESCRIPTION OF THE FIGURE

The FIGURE is a representation of the inventive method. The method first selects from the antibody phage display the specific antibody that binds to a first protein of interest in the proteome matrix. This phage antibody is then exposed to another phage population that has been engineered to display on its surface the proteome of the cell. The phage selected by the antibody displays either the first protein or a second protein containing an idiotypic region of the first protein in the proteome matrix but now in sufficient amounts to permit positive identification.

### DETAILED DESCRIPTION OF THE INVENTION

The invention will now be described in detail by way of reference only using the following definitions and examples. All patents and publications referred to herein are expressly incorporated by reference.

The inventive method is useful for the identification and characterization of proteins, especially small peptides, that are biologically active from a tissue of interest. Additionally, precursor proteins may also be identified and characterized. The tissue is processed such that a mixture of cellular proteins is obtained. The protein mixture may be fractionated or proteins separated by size by conventional techniques. Such techniques include, but are not limited to, column filtration, chromatography and the like. The protein mixture is then run on two dimensional gel electrophoresis. The protein of interest is isolated from the gel and contacted with an antibody-phage display library to yield at least one high-affinity antibody phagemid to the protein of interest. The antibody-phagemid is then used to screen a cDNA phage display library displaying the proteome of the tissue of interest. Thus, the high-affinity antibody recognizes a protein displayed in the cDNA phage library that possesses an epitope found on the protein of interest from the protein mixture, i.e., a protein-specific phagemid. The cDNA insert associated with the peptide-specific phagemid can then be used to identify the protein of interest using techniques well-known in the art, such as PCR, DNA sequencing, and the like.

The present invention provides a method of identifying proteins isolated on a proteomic gel or matrix. The key step is to isolate from a phagemid display antibody library, specific antibodies that recognize single protein spots on a proteome gel. The phagemid is then used for two complementary protocols. First, the expressed antibody is used to screen a separate phagemid display library expressing total cellular proteins and the protein antigen identified from the sequence of the cDNA spliced into the phagemid genome. Second, the DNA encoding the antibody is subcloned into a mammalian expression vector and expressed in a fibroblast where it accumulates and neutralizes the target antigen in vivo. The effect of the “protein knockout” is assessed by the ability of the cell to respond to insulin-like growth factor's (IGF) mitogenic and anti-apoptotic signals. This strategy however is generally applicable to the analysis of signaling pathways for many other growth factors in addition to IGF.

Definitions

As used herein, the following terms or abbreviations, whether used in the singular or plural, will have the meanings indicated:

“Antibody” means an immunoglobulin that specifically binds to and is thereby defined as complementary with a particular spatial and polar organization of another molecule such as the unknown protein to be identified by the inventive method described herein. Antibodies may include a complete immunoglobulin or fragment thereof, which immunoglobulins include the various classes and isotypes, such as IgA, IgD, IgE, IgGI, IgG2a, IgG2b and IgG3, IgM, etc. Fragments thereof may include Fab, Fv and F(ab′)₂, Fab′, and the like. In one aspect, the antibody is displayed in a phagemid library.

The term “biopanning” as used herein refers to by an in vitro selection procedure. In its simplest form, biopanning is carried out by incubating a pool of phage-displayed variants with a target of interest that has been immobilized on a plate or bead, washing away unbound phage, and eluting specifically bound phage by disrupting the binding interactions between the phage and target.

The eluted phage may then be amplified in vivo and the process repeated, resulting in stepwise enrichment of the phage pool in favor of the tightest binding sequences. Repetition of the biopanning and amplification process will generally result in a small (e.g. less than 10) high affinity phagemids for a given protein.

“Idiotype” or “idiotypic” is a unique motif within a protein that gives rise to an antigenic determinate. The motif may be present on more than one protein. The term may be used interchangeably with epitope herein.

“Tissue” includes whole tissue, single cells and sub-cellular fractions thereof, including extracts and isolates thereof. For example, a tissue fluid, such as brain microdialysate, is included within the definition of tissue.

As used herein, “phage display” describes an in vitro selection technique in which a peptide or protein is genetically fused to a coat protein of a bacteriophage, resulting in display of the fused protein on the exterior of the phage virion, while the DNA encoding the fusion resides within the virion. This physical linkage between the displayed protein and the DNA encoding it allows screening of vast numbers of variants of the protein, each linked to its corresponding DNA sequence, by a simple in vitro selection procedure called “biopanning” (see definition above).

Therefore, in the inventive method an antibody phage display and a protein phage display, among others, are contemplated. An antibody phage display would comprise the DNA for an antibody and would display the antibody on the exterior of the phage. Similarly, a protein phage display comprises the DNA for a protein and displays the protein on the exterior of the phage.

A “phage display library” is a collection of phage displays exhibiting a certain characteristic. Thus, an antibody phage display library would include a collection of-unique antibodies each displayed on the exterior of a phage. Similarly, a cDNA phage display library would include the proteome of a tissue with each protein displayed on the exterior of a phage.

“PCR” or “polymerase chain reaction” means a technique, well-known in the art, for reproducing specific DNA sequences in vitro. The sequence of PCR involves the following steps:

A: The DNA to be reproduced is heated to separate the two template strands.

B: Two primers which are complimentary to the region to be amplified are added.

A heat-stable DNA polymerase enzyme is also added. The enzyme catalyses the extension of the primers, using the DNA strand as template. The solution is heated to break the bonds between the strands of the DNA. When the solution cools, the primers bind to the separated strands, and DNA polymerase quickly builds a new strand by joining the free nucleotide bases to the primers. When this process is repeated, a strand that was formed with one primer binds to the other primer, resulting in a new strand that is restricted solely to the desired segment. Thus, the region of DNA between the primers is selectively replicated. Further repetitions of the process can produce billions of copies of a small piece of DNA in several hours.

The cycle is repeated, with the newly synthesized double stranded DNA being heat-denatured and the enzymes extending the primers attached to the liberated single DNA strands. The chain reaction, once set up, results in the exponential amplification of the original DNA, where the number of cycles (n) determines how many copies of the DNA (2ⁿ) are produced.

The term “proteome” is a portmanteau word, blending the words protein and genome. The “proteome” is simply the protein complement expressed by a genome or tissue. The concept of the proteome is fundamentally different to that of the genome: while the genome is virtually static and can be well-defined for an organism, the proteome continually changes in response to external and internal events. For example, E. coli will express different proteins when cultivated with minimal media instead of complete media. Therefore, its proteome will be different and so have a different proteome. Similarly, during mammalian development cells express different proteins, develop dissimilar but characteristic proteomes, and ultimately differentiate into various tissues.

Methods

In proteome projects, one of the primary goals is to separate and visualize as many proteins from a sample as possible, thus allowing them to be catalogued by computer and studied by analytical techniques. The inventive method is based on two well-established methodologies that have been combined in an innovative way to create a technology platform with unparalleled potential for research and biotechnology.

Two Dimensional Gel Electrophoresis

The complete spectrum of proteins expressed by a cell or tissue can be visualized by two dimensional gel electrophoresis (2DE) in which they are separated by isoelectric focussing in a first dimension and then in a second dimension according to apparent molecular weight by SDS-PAGE. See Klose (Human Genetik 26: 231-24, 1975) and O'Farrell (J. Biol. Chem. 250: 4007-4021, 1975). The 2D array thus generated may contain up to 10,000 protein “spots” on a single gel. Therefore, in principle it is possible to resolve the entire proteome on a single two-dimensional gel. 2-D PAGE is one of the most efficient and powerful methods for purifying proteins in small quantities.

Two-dimensional gel electrophoresis has been used successfully to identify differences in proteins in a wide variety of normal and pathological states. To exploit the significance of these differences however requires a knowledge of the identity and function of the proteins identification by conventional means (e.g., N-terminal sequencing and mass spectroscopy) is limited by the small amounts of material in each spot and invariably this relies on matching the partial sequence to an existing protein data base. An ambiguous or novel sequence requires the laborious process of back-reference to a cDNA library for complete characterization. Thereafter, the functional significance is usually assessed by protracted and often unpredictable gene knock out or antisense methods. The inventive method disclosed herein describes a superior alternative based on phage display libraries which is capable of ultra-sensitive, high through-put screening of proteins of interest.

Immobilized pH gradients (IPG) (see Bjellqvist et al., J. Biochem. Biophys. Meth. 6: 317-339, 1982 and Gorg et al., Electrophoresis 9: 531-546, 1988) can now be used for the pH range 3 to 12 and have become the method of choice for the isoelectric focusing. IPG gels do not suffer from cathodic drift and focus proteins to equilibrium, thus providing very high reproducibility. Furthermore, IPG gels are commercially available (Amersham Pharmacia Biotech), so the reproducibility among different laboratories needed to establish and standardize two-dimensional gel databases is now possible. When very high resolution is required, the pH range of IPG gels can be narrowed, for example, one pH unit spans 16 cm, allowing us to “zoom in” on a pH range of interest. An important feature for using IPG gels in the first-dimension is their ability to accommodate the high sample loads needed for micropreparative 2-D PAGE. Current methods for sample loading produce hundreds to thousands of protein spots on a single gel, with the quantities of each protein ranging from high nanogram to low microgram amounts. The study of very low-abundance proteins by 2-D PAGE is still challenging, even after loading milligram quantities of samples. Prefractionation either by sub-cellular compartmentalization or by narrow-range micropreparative 2-D PAGE often helps. However, a method of identifying very low-abundance proteins remains desirable.

Phage Display

A basic component in this technology is a phage antibody display library. Such libraries are commercially available from vendors/licensors such as Cambridge Antibody Technology (CAT). In engineering their library CAT fused the variable regions of human immunoglobulin heavy and light chains to the gene for a minor coat protein of the M13 bacteriophage. Further mutations were subsequently introduced in vitro into the variable regions to generate a repertoire of antibodies capable of recognizing a very large number (for example, more than 10¹²) of epitopes. Each phage particle contains a single antibody gene in its genome and displays on its surface a unique monoclonal antibody that can be selected from the vast number of other phage in the library by its ability to bind to a specific protein epitope. This library has sufficient antibody diversity to recognize the 10,000 or so proteins on a 2DE gel many times over and therefore has immense potential for adaptation and application to the problems of multiple protein analysis. Moreover, the phage infect and multiply in E. coli and are readily handled by standard microbiological procedures under PC1 containment facilities. The generation of such a library is described, e.g., in McCafferty et al., Nature, v.348, pp. 552-554 (Dec. 6, 1990); and a person of ordinary skill in the art will be able, having regard to that skill and the literature available, to generate a similar library.

Ribosome Display

“Ribosome display” is a novel method which has been developed in which whole functional proteins are enriched in a cell-free system for their binding function, without the use of any cells, vectors, phages or transformation (Proc. Natl. Acad. Sci. 94, 4937, 1997; Curr. Opin. Biotechnol. 9, 534, 1998; Curr. Top. Microbiol. Immunol., 243, 107, 1999; J. Immunol. Meth. 231, 119, 1999; FEBS Lett., 450, 105, 1999). This technology is based on in vitro translation, in which both the mRNA and the protein product do not leave the ribosome. This results in two fundamental advantages: (i) the diversity of a protein library is no longer restricted by the transformation efficiency of the bacteria, and (ii), because of the large number of PCR cycles, errors can be introduced, and by the repeated selection for ligand binding, improved molecules are selected. Correctly folded proteins can be selected, if the folding of the protein on the ribosome is secured (Nat. Biotechnol. 15, 79, 1997).

In this method the whole cell cDNA is subjected to a transcription and translation process using established methods. The mRNA read off the cDNA is such that it lacks a termination codon and so is translated into protein on the ribosome surface and also remains attached to it. See Hanes et al., Proc. Natl. Acad. Sci. 94:4937 (1997).

Briefly, a library of DNA is transcribed in vitro. It is then translated under conditions where the protein and the mRNA stay on the ribosome, and such that the protein folds correctly. This whole complex, consisting of mRNA, folded protein and ribosomes can then be bound to immobilized ligand (or alternatively, be recognized by an antibody). From these immobilized complexes, the mRNA is isolated.

Screening techniques other than PCR, RT-PCR or hybridization are well known to those of skill in the art and the selection of the techniques does not limit the present invention. The procedures for isolating and identifying gene fragments are well known to those of skill in the art; see, e.g. T. Maniatis et al, Molecular Cloning (A Laboratory Manual), Cold Spring Harbor Laboratory Press.

Once identified and sequenced, the nucleotide fragments of the cDNA insert may be readily synthesized by conventional means such as solid phase oligo-DNA synthesis (Letsinger et al., (1965) Oligonucleotide synthesis on a polymer support. J. Am. Chem. Soc. 87:3526-3227). Alternatively, the DNA may be produced by recombinant methods, then sequenced. Cloning procedures are conventional and are described by T. Maniatis et al, Molecular Cloning (A Laboratory Manual), Cold Spring Harbor Laboratory (1982).

General Strategy

The novel application of the antibody-phagemid display library is outlined in the FIGURE with respect to proteins isolated from a 2DE gel. There are three basic steps:

1. Isolation of a Phage Antibody or a Candidate Protein.

An isolated protein of interest from a tissue of interest is “biopanned” against the antibody-phagemid display library. The protein of interest may be any protein, known or unknown, that has been purified and transferred to a support matrix (e.g. a protein electroblotted from a 2DE gel onto a nylon membrane) or immobilized by any technique that does not effect its conformation or function (e.g. a protein separated by HPLC and immobilized on beads or in the wells of microtiter plates). Phage that bind to the protein with high specificity are recovered and the procedure repeated as many times as necessary (typically 2-3 times) to isolate antibody phages of maximum specificity for the protein. Desirably less than 10 high-affinity antibody phages are isolated. These are then replicated in bacteria to give a much reduced subset of phage antibodies against the candidate protein. (The FIGURE also shows an intermediate PCR step for amplifying bound phage particles in the event that additional amplification is necessary.)

2. Identification of the Structure of the Candidate Protein.

A second phage library is prepare containing the cDNAs from the proteome of the tissue of interest using technique similar to those discussed above. In this case the phage library displays on their surface all the different proteins expressed in the cell, one protein per phage. The selected phage antibody subset is then screened against this library and the bound phage purified. The proteins displayed on these phage(s) should be the same as or contain an idiotypic region of the original protein. Individual phages are isolated and the sequence of the displayed protein deduced from the sequence of the cDNA in the phagemid genome. Thus a combination of phage antibody specificity and an ability to amplify individual phage isolates in bacterial allows for complete characterization of virtually any protein. In the case where the original protein is of higher molecular weight, the protein(s) found by this technique may well be the same as the original protein of interest; in the case where the original protein is of lower molecular weight, such that it can be isolated and sequenced by conventional techniques, the protein(s) found by this technique may well include proteins that are idiotypic to the original protein and may be precursors to the original protein.

3. Identification of the Function of the Candidate Protein.

Among the 10-20 phage antibodies isolated against a protein in step 1 there is a high probability that at least one will not only recognize the protein but also neutralize its biological action in vivo. A strategy that reveals this function and which circumvents the disadvantages of conventional knock out experiments is to transiently express the neutralizing antibody gene in vivo by transfection into cells and to screen for effects on general cellular responses (e.g., viability, hormone/growth factor responses, cell cycle characteristics, differentiation) using standard procedures. For example, in the case where the original spot was seen to increase in response to a given hormone, this strategy would both identify the protein and also reveal whether it is essential for hormone action and perhaps also suggest a particular mechanism of action.

Experimental Outline

While all the basic techniques outlines above are individually well-established, their integration into a discovery platform is innovative and has never before been attempted. This can be achieved in a straightforward manner by analyzing a spot whose identity is already established or by “spiking” a 2DE gel with an appropriate amount of a known protein. An example of the latter is phospholipase Cβ(PLCβ) which we have shown to be essential for the mitogenic action of insulin-like growth factor (IGF-I). An experimental plan would be as follows:

1) Recombinant PLCβ is made by the baculovirus expression system—a standard procedure.

2) Total cellular proteins from Swiss 3T3 fibroblasts are spiked with program amounts of recombinant PLC and its position identified on a 2DE gel. (Achieved by labeling with ³H-methionine.)

3) The PLC spot is electro-transferred to a nylon membrane and biopanned with the CAT phage antibody library. The bound phage particles are purified by several rounds of biopanning. The result is a PLC-specific phage.

4) A second phage library displaying total 3T3 cell proteins is screened with the PLC-specific phage and cross-reacting phage purified.

5) Individual phage are isolated, their cDNA inserts sequenced and compared with the known DNA sequence of PLCβ.

6) Antibody expressing DNA from phage isolated in step 3 are cloned into a mammalian expression vector and transiently transfected into Swiss 3T3 cells. After 24 hours, the cells are challenged with IGF-I and the mitogenic response measured by BrUdr incorporation into DNA. Some of the phosphoproteins have similar MWs but different pIs, suggesting that there are multiple phosphorylated isoforms of a single protein.

Experimental Background

Cellular protein profiles, i.e., the proteome, are altered in disease such as cancer and with changes in the external milieu. For example, it is known that insulin-like growth factor (IGF), as well as other growth factors, hormones, neurotransmitters and the like, effect the cell in unique ways. While changes in the proteome can be monitored it is not always known which proteins are altered.

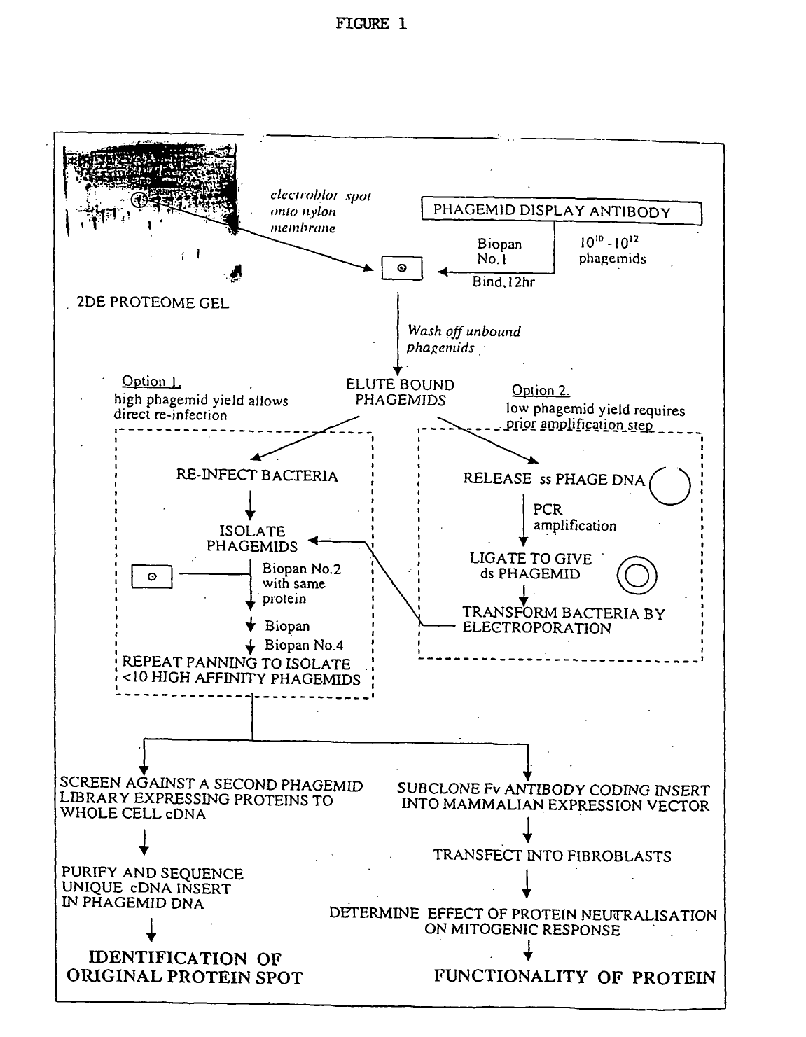

The dynamics of nuclear protein phosphorylation events following IGF-I stimulation is investigated. Quiescent Swiss 3T3 cells are radiolabelled with ³²Pᵢ and treated with IGI-I for different periods (5 min, 10 min, 15 min, 20 min, 30 min, and 60 min). The nuclei from these cells are then purified in the presence of protease and phosphatase inhibitors, and nuclear proteins are resolved by analytical 2-DE as described for FIG. 1. The phosphoproteins are detected by autoradiography or phosphorimaging, and systematically analyzed using Imagemaster™ 2D software to detect proteins whose phosphorylation/dephosphorylation is induced by IGI-I.

Once these nuclear IGF-I-responsive phosphoproteins (NIRPPs) are detected, preparative 2-DE is performed to isolate sufficient amount of them for further characterization. To this end, up to 5 mg of nuclear proteins is applied to IPG strips by direct rehydration (in an aqueous solution containing all necessary additives: 8 M urea, 0.5% non- or zwitterionic detergent, 2% thiol reagent, and 0.5% carrier ampholytes) using a methacrylate rehydration chamber (Rabilloud et al., Electrophoresis 15: 1552-1558, 1994), and then resolved by 2-DE as above. The resolved nuclear proteins are visualised by staining with Coomassie Brilliant Blue (CBB). The NIRPPs in the gels are located by matching the CBB-stained gels with their corresponding autoradiograms. These proteins are subsequently excised from the gels and in-gel digested by trypsin. The tryptic digests are separated by reversed phase high performance liquid chromatography (RP-HPLC), and the well-resolved peptides are chosen for amino acid sequencing. The sequence information is used to determine the identity of the protein by reference to a database. If the sequence cannot be found in the database, the information is used to design degenerate PCR primers to clone the gene for the protein.

The approach described above allows the identification of at least those NIRPPs with high or moderate abundance. For those low abundance NIRPPs which cannot be directly visualised by CBB staining, the following strategies are employed to enrich and identify these proteins: (I) To use extreme narrow pH range IPG gel strips. These recently-developed gel strips, which are 18-cm long with pH interval of as small as 0.5, allow up to 50 mg proteins to be loaded on a single gel, and thus greatly increase the chance to detect low-abundance NIRPPs (http://www.apbiotech.com). (II) Prior to 2-DE, pre-fractionation of nuclear protein samples using Gradiflow (Gradipore, Ltd., French's Forest NSW 2086, Australia; see also http://www.gradiflow.com). This new preparative electrokinetic membrane apparatus was designed to fractionate proteins according to their relative mobility under controlled electrophoretic conditions (Gradiflow Technical Overview, May 1999). (III) Post 2-DE enrichment. Following 2-DE separation, low-abundance NIRPPs are collected from their ‘spots’ from multiple gels, pooled and refractionated, thus allowing “in-gel” concentration. (IV) Alternatively, low-abundance NIRPPs are visualised by modified silver staining. This new method is about 100-fold more sensitive than CBB staining. Following modified silver staining, proteins are identified either by direct amino acid sequencing or by peptide mass fingerprinting using matrix-assisted laser desorption/ionisation time of flight mass spectrometry (MALDI-TOFMS). (V) It is intended to use the inventive phagemid display strategy described herein in tandem with that outlined above to extend the range of sensitivity of detection to the low abundance proteins.

To Elucidate the Signaling Pathways Underlying the Phosphorylation and/or Dephosphorylation of those NIRPPs Identified Above.

(a) Studies with Specific Signaling Inhibitors.

As discussed above, IGF-I signals mainly through two separate kinase cascades namely the Ras-Raf-MAPK and PI-3-kinase-PDK-PKB cascades. Recent Studies revealed that these two signaling cascades cross talk with each other. In addition, IGF-I induces activation of nuclear PLC-βI and production of nuclear DAG which in turn stimulates the translocation of PKC-α to the nucleus. These signaling events can be specifically blocked by different kinase inhibitors, such as PI-3-kinase inhibitor wortmannin, MAPKK inhibitor PD98059 and PKC inhibitor calphostin C. These specific inhibitors are used to investigate which signaling pathway is involved in the phosphorylation events of NIRPPs. To this end, the ³²P-labeled 3T3 cells are pre-incubated with one of these inhibitors before IGF-I stimulation, and phosphorylation of nuclear proteins is subsequently analyzed as above, to see whether the action of IGF-I on NIRPPs is blocked under these circumstances. These studies also distinguish protein targets which are phosphorylated directly by MAPK from those phosphorylated indirectly via MAPK-dependent activation of PKC.

(b) Mapping the Precise Phosphorylation Sites of NIRPPs.

It is well established that the substrate specificity of protein kinases is determined by the consensus sequence motif surrounding the phosphorylation site. Therefore, identification of phosphorylation sites could provide important clues for determining the nature of the protein kinases that are directly responsible for the phosphorylation of a particular protein. Nuclei from ³²P labeled 3T3 cells treated with IGF-I are separated by 2-DE, and the proteins visualised by autoradiography as above. The ‘spots’ corresponding to NIRPPs are excised and pooled from multiple gels. Aliquots of these proteins are subjected to phosphoamino acid analysis to determine which type of amino acid residues (serine, theronine or tyrosine) is phosphorylated. The remaining samples are digested by proteases such as trypsin, and the protease digests are separated by RP-HPLC. Each fraction is collected, and the fractions that contain ³²P-labeled phosphopeptides are detected by liquid scintillation counting. The precise phosphorylation sites of these purified phosphopeptides is consequently analyzed by a phosphoamino acid releasing assay using solid phase Edman degradation sequencing. Alternatively, for those low abundance NIRPPs, the protease digests are directly analyzed by MALDI-TOF MS. The number of phosphate residues and their phosphorylation sites are determined by comparing the experimentally observed mass with the theoretically generated mass profiles for all the known protein sequences present in a database (http://www.expasy.hcup-h.ch).

To Establish the Role of the IGF-I Responsive Phosphoproteins in Cell Growth and Proliferation Using a Double Phagemid Display Strategy.

For proteomics to be biologically meaningful requires a prior knowledge of an identified protein's function or a means of readily determining its functional relevance in vivo. Most of the functional strategies are DNA based (e.g., gene knock out, antisense RNA), are technically complex and are unpredictable in outcome. Here we use a protein based approach which exploits phagemid display to give a high-sensitivity, high through-put screen for structure/functional relationships of proteins identified by proteome analysis. The key step is to isolate from a phagemid display antibody library, specific antibodies which recognize single protein spots on a proteome gel. The phagemid is then used for two complementary protocols. Firstly, the expressed antibody is used to screen a separate phagemid display library expressing total cellular proteins and the protein antigen identified from the sequence of the cDNA spliced into the phagemid genome. Secondly, the DNA encoding the antibody is subcloned into a mammalian expression vector and expressed in a fibroblast where it accumulates and neutralizes the target antigen in vivo. The effect of the “protein knockout” is assessed by the ability of the cell to respond to IGF's mitogenic and anti-apoptotic signals. The strategy is summarized in the FIGURE.

### EXAMPLES

The following preparations and examples are given to enable those skilled in the art to more clearly understand and to practice the present invention. They should not be considered as limiting the scope of the invention, but merely as being illustrative and representative thereof.

### Example 1

Screening the Proteome with a Phage Display Antibody Library

A phagemid display library which expresses murine immunoglobulin V_{H} and V_{L} variable genes as a single F_{V} fragment fused to the N-terminus of gene III of an fd phage vector is used. The V gene regions have been hypermutated in the antigen binding loops to generate a repertoire of antibody specificities capable of recognizing an infinite variety of epitopes. These are displayed on the phage surface (4×V regions/virion) as monoclonal antibodies and can be selected from the library by screening with an appropriate antigen. This technology was first described for human V genes in 1990 (McCafferty et al. Nature 348:552, 1990). An equivalent murine library, produced by Pharmacia, is now in the public domain and therefore offers a unique opportunity to couple the enormous potential of proteomics with the unparalleled discrimination of phagemid display.

Spots of interest from 2DE gels are electroblotted onto nylon membranes and “biopanned” with 10¹⁰-10¹² phagemid particles. The membrane is washed free from unbound phagemid and the bound virions eluted with glycine buffer at pH 2. Two options can then be deployed depending on the number of phagemids recovered.

Option 1: If the phagemid number is high enough, they are used to re-infect E. coli directly and the biopanning procedure repeated with the same protein until a small number of phage with high binding efficiency are obtained. The original paper of McCafferty et al. reported that two rounds of biopanning enrichment were sufficient to select a single copy of a test phagemid antibody in the presence of 4×10⁶ wild type phagemids thus demonstrating the exquisite sensitivity and specificity of the method. Given adequate recovery of bound phagemids from membranes this strategy is successful for most 2DE spots; however, where phagemid numbers are too low for direct re-infection an alternative option is proposed.

Option 2: The pooled single stranded DNA of the phagemid virions are amplified by PCR using forward and reverse primers corresponding to the 5′ and 3′ termini of the phagemid DNA. If the primers are phosphorylated at their 5′ ends it is then possible to ligate the PCR product to form double stranded phagemid DNA with which to transduce E. coli by electroporation. Subsequent infection with a helper phagemid (i.e., one not containing a gene III fusion) promotes virion packaging and results in an amplified population of the original bound phagemid with which to carry out the enrichment procedure as descried in Option 1.

### Example 2

Use of a Phagemid Monoclonal Antibody to Identify the Original 2DE Protein Spot

A cDNA library is made from Swiss 3T3 total cell mRNA and screened by the phage display method. The library is constructed in the phage display vector pJuFo following an already established method (Crameri and Suter, Gene 137: 69-73, 1993). pJuFo utilizes modified leucine zipper domains of Jun and Fos which couple covalently in the periplasm of the host to permit library proteins to be fused both C-terminally to a vector encoded peptide and N-terminally to the filamentous phagemid cIII coat protein. Helper phage infection releases phage particles each displaying on their surface the protein encoded by the particular cDNA within. These are screened by biopanning with phagemid display antibodies conjugated to biotin and strepavidin coated microtitre wells by the phage ELISA method and the effectiveness of the biopanning is monitored by the titre of the phagemids recovered at successive rounds. After isolation, phage clones are sequenced and sequences compared with those in the GenBank and EMBL databases to identify the cDNA. Confirmation that the sequence corresponds to the original 2DE spot comes initially from molecular weight estimation, the partial protein sequence derived from MALDI-TOF analysis of tryptic peptides and N-terminal Edman sequencing.

Changes in phosphorylation patterns of nuclear proteins in Swiss 3T3 cells following IGF-I stimulation. Confluent Swiss 3T3 cells in 100 mm dishes were starved in serum-free DMEM for 24 hours and were then incubated in DMEM without sodium phosphate for another one hour to deplete the ATP metabolic pool. The cells were subsequently incubated with 0.2 mCi/ml ³²Pi for 4 hours, and treated without or with 40 ng/ml IGF-I for 5 minutes. Nuclei were then purified and solubilised in lysis buffer [50 mM Tris, pH 8.0/10 mM EDTA/1% (wt/vol) SDS plus protease inhibitors leupeptin, pepstatin, and PMSF all added at 0.2 mM]. Nuclear lysates with equivalent amount of radioactivity were applied to immobilised pH-gradient strips (IPG) with pH range of 4-7 for isoelectric focusing, and then separated by 12-14% gradient SDS-PAGE. The nuclear phosphoproteins were then visualised by autoradiography and analyzed by image software.

### Example 3

Use of Phagemid Monoclonal Antibody to Assess Functional Relevance of Proteins

Over-expression of normal or mutated cDNA in a cell line is often used to assess protein function. A criticism of this approach is that abnormally high levels of an endogenous protein, especially if it has a regulatory function, may distort any conclusion about its action. Introduction of antisense sequence to the protein of interest into cells is also unpredictable and complete ablation of synthesis is often difficult to achieve.

An alternative strategy is to exploit cloned phagemid antibodies to known proteins to neutralize their function in vivo. This can be done by recovering the Fv coding region in the phagemid DNA by restriction enzyme digestion and subcloning it into a mammalian expression vector (e.g. pcDNA3). It may also be efficacious to fuse a nuclear localization sequence (NLS) to the Fv gene to target the antibody to the nucleus and a green fluorescent protein (GFP) gene under control of a separate promoter to allow visualization of transfected cells. The vector is introduced into 3T3 fibroblasts and its IGF responsiveness determined. Two strategies are used:

A. High Through-Put Screening of Transiently Transfected Cells.

In transient transfection not all cells in a culture take up and express the introduced gene therefore the functional needs to be applicable at the single cell level. Cells on coverslips are first transfected with the NLS-GFP-Fv vector and two days later transferred to serum free medium for 16 hrs. Cultures are stimulated with IGF-I for a further 16 hrs and then pulsed with 5′BrdUrd for 10 mins. Cells are then permeabilized with detergent to permit the entry of a monoclonal antibody against 5′BrdUrd and a Cy5-conjugated anti-mouse IgG. Fluorescent analysis is carried out at wavelengths specific for GFP and Cy5. Functional impairment of IGF's mitogenic response is scored for cells which fluoresce for GFP (and therefore express the Fv antibody) but not for Cy5. Cells which do not take up the vector will fluoresce for Cy5 only and act as a positive control for IGF stimulation. In this way phagemid antibodies to multiple 2DE protein spots can be screened relatively rapidly for their functional relevance to the IGF-dependent mitogenic response.

B. Analysis of Mechanism in Stably Transfected Cells.

To further determine the mode of action of proteins of interest highlighted by the above screen it is necessary first to derive clonal cell lines expressing the antibody in a stable manner. This is achieved by selecting cells in the presence of the drug G418, resistance for which is built into the pcDNA3 expression vector. If the assumption that signals are propagated by sequential protein phosphorylations is correct, neutralization of an individual signaling molecule will have a marked effect on the phosphorylation of subsequent molecules in the cascade. Thus, a broad understanding of these intermolecular relationships can be obtained by comparing the iterative proteome profile of nuclear phosphoproteins from normal and antibody expressing cell lines and identifying the differences. From this information it is possible to piece together the sequence of very early signaling events which some hours later lead to the initiation of the cell cycle.

### Example 4

To Characterize the Dynamic Changes in Phosphorylation/Dephosphorylation of Nuclear Proteins Following IGF-I Stimulation.

As discussed above, several important protein kinases which play a central role in IGF's biological action, are translocated to the nucleus following IGF-I stimulation. However, the details about how these kinases exert their actions in the nucleus and eventually control cell cycle and proliferations are still poorly characterized. Surprisingly, little is known about their physiological nuclear targets.

Proteome analysis is used to uncover a complete display of protein phosphorylation events that occur at the nuclei following IGF-I stimulation, and to search for novel nuclear targets of this growth factor. The central technique of proteome analysis is two dimensional gel electrophoresis, in which the proteins are separated firstly by isoelectric focusing according to their isoelectric points (pI) and then by SDS-PAGE according to their molecular weight in a second perpendicular dimension. The generated two-dimensional array of proteins may contain up to 10,000 protein ‘spots’ in a single gel. This technique is unique in its ability to separate protein isoforms with subtle pI value differences (as small as 0.01), and thus serve as an exceptional tool to display proteins with multiple phosphorylated isoforms.

Proteome analysis has been employed successfully to identify differentially expressed proteins under pathological conditions and to investigate the protein.

### Example 5

Construction of Ribosome Display Library

mRNA is extracted from about 1-5×10⁶ cells and transcribed to cDNA. After PCR amplification, PCR products are purified by agarose gel electrophoresis and extracted from the gel with the QIAEX gel extraction kit (Qiagen). An assembly PCR is carried out (see Krebber et al., J. Immunol. Methods 201:35-55, 1997) and the PCR products are directly diluted 3-fold in SfiI reaction buffer, digested with SfiI and separated by using agarose gel electrophoresis. The cut DNA is extracted from agarose gels by Amicon spin columns, concentrated by isopropyl alcohol precipitation and dissolved in sterile water. Purified PCR products are ligated in a 30-μl reaction mixture with SfiI-cut vector pAK200 overnight at 16° C. (molar ratio insert to vector=1:2). To introduce the features necessary for ribosome display, the ligation mixtures are directly amplified in two steps by PCR, by using in the first step the primers SDA, which introduced a ribosome binding site, and T3Te, which encodes the translated early transcription terminator of phage T3, and in the second step primers T3Te and T7B, which introduced the T7 promoter as well as the 5′-loop (Hanes et al., PNAS 91:4937-4942 (1997)). PCR products are used without purification for in vitro transcription, and RNA is purified by LiCl precipitation.

### Example 6

In Vitro Translation of mRNA

In vitro translations in an E. coli S-30 system are performed as described by Hanes et al (supra) with small modifications. Briefly, the in vitro translation is carried out for 8 minutes at 37° C. in a 220-μl reaction that contained the following components: 50 mM Tris·HOAc, pH 7.5, 30 mM NH₄HOAc, 12.3 mM Mg(OAc)₂, 0.35 mM of each amino acid, 2 mM ATP, 0.5 mM GTP, 1 mM cAMP, 0.5 mg/ml E. coli tRNA, 20 μg/ml folinic acid, 100 mM KOAc, 30 mM acetylphosphate, 1.5% polyethylene glycol 8000, 3.5 μg/ml rifampicin, 1 mg/ml vanadyl ribonucleoside complexes, 3.5 μM anti-ssrA oligonucleotide, 0.3 μM protein disulfide isomerase, 51.4 μl of E. coli MRE600 extract and 90 μg/ml of mRNA. The anti-ssrA oligonucleotide is necessary to prevent the nascent protein from being released from the ribosome.

### Example 7

Phagemid Vector Construction for Proteome Display

The phagemid vector, pJuFo, is constructed to fuse the modified Jun Leu zipper with the C-terminal domain of the pIII protein of the filamentous phage. The cloning system allows the expression and enrichment of functionally folded cDNA products covalently linked to the filamentous phage, and thus to the genetic information required for their production. At the N- and C-termini of the original Jun Leu zipper, Cys residues are added via GlyGly spacers (O'Shea et al., Science 245:645-648 (1989)). The Jun::pIII fusion is placed under the control of a lac promoter/operator element and directed to the periplasmic space by the pelB signal sequence. Gene products to be captured on the outer phage surface are linked to the C-terminus of the modified Fos Leu zipper, which is flanked by Cys residues added via GlyGly spacers at the N- and C-termini. The genes to be coexpressed with Fos are cloned as BglII-XbaI, BglII-KpnI or XbaI-KpnI fragments and placed under the control of a separate lac promoter/operator element and directed to the periplasmic space of the E. coli host by the pelB signal sequence. The periplasmic space allows functional assembly of Jun/Fos heterodimers as parallel coiled-coil structures followed by proper disulfide bond formation mediated by the engineered Cys residues flanking the Leu zippers. During helper phage superinfection the Fos-fusion protein captured by Jun::pIII is incorporated into the virion. Purified phage displaying gene products on the surface are used for further investigations.

DNA encoding the Leu zippers from Jun and Fos genes is isolated by PCR using the original constructs (Roche Research Center, NJ, USA) as templates with 5′- and 3′-primers. PCR amplifications are performed in a thermocycler using standard protocols (Sambrook et al., Molecular Cloning: A Laboratory Manual. 2^{nd} Edition. Cold Spring Harbor Laboratory Press. Cold Spring Harbor, N.Y. 1989) and amplification reagents obtained from Anawa (Wangen, Switzerland). In a first step the Jun-encoding gene fragment is inserted in-frame 5′ to the pill coding sequence of pComb3 as a HindIII-BamHI fragment. For cloning of DNA encoding the modified Fos Leu zipper region, phagemid DNA containing the modified Jun zipper is digested with SacI+XbaI, gel-purified, extracted with glass milk (Bio 101, La Jolla, Calif., USA) and ligated with Fos PCR product digested with SacI+XbaI. The ligation mixture is used to transform 50 μl of E. coli XL1-Blue (Stratagene, La Jolla Calif., USA) electrocompetent cells which are then plated on LB/Ap plates as described by Barbas et al., Comp. Methods Enzymol. 2:119-124 (1991). Single colonies are picked, grown in liquid culture, DNA is prepared (see Holmes et al., Anal. Biochem. 114:193-197 (1981)) and the final construct is verified by restriction analysis. The pJuFo is recovered from the liquid cultures by PEG/NaCl precipitation and stored at −20° C. until use.

Construction of the pJuFo:proteome cDNA is briefly as follows: the cDNA is subcloned into pJuFo, expressed, and the Fos-proteome fusion proteins captured on the surface of phages. Recombinant phage particles are used to infect 20 ml of E. coli XL1-Blue cells together with 10¹¹ pfu of R408 helper phage. After incubation for 15 minutes at 37° C., 200 ml 2×YT medium (Barbas et al., PNAS 88:7978-7982, 1991) containing 100 μg/ml ampicillin are added and incubation continued for 6 hours. The culture is then heated at 70° C. for 20 minutes then centrifuged for 10 minutes at 8,000×g. The decanted supernatant containing the in vivo excised cDNA library into the pBluescript phagemid was stored at 4° C. To prepare DNA excised phagemid (10¹⁰ pfu) is used to infect E. coli XL1-Blue (10 ml, A₆₀₀=1). After addition of 250 ml LB-medium containing 100 μg/ml ampicillin and further incubation at 37° C. overnight, plasmid DNA was prepared using a commercial kit (Diagen, Dusseldorf, Germany). For the construction of a phage cDNA library, 2 μg of this DNA is cleaved with XbaI+KpnI, the inserts gel purified and ligated to 4 μg of pJuFo vector digested with XbaI+KpnI. The ligation mixture is used to transform E. coli XL1-Blue cells by electroporation and a phage expression vector is prepared by helper phage infection as described by Barbas and Lerner (1991, Comp. Methods Enzymol. 2: 119-124).

Although the foregoing invention has been described in some detail by way of illustration and example for purposes of clarity and understanding, it will be obvious that certain changes and modifications may be practiced within the scope of the appended claims.

We claim:

1. A method of identifying a first protein, or a second protein containing an idiotypic region of the first protein, from a tissue of interest by double phage screening, said use comprising:
(a) contacting said first protein with an antibody phagemid display library to form a complex between said first protein and at least one member of said library;
(b) screening a cDNA phagemid display library of the proteome of the tissue of interest with the complex-forming antibody phage to identify a protein-specific phagemid that displays a second protein that binds the antibody phagemid; and
(c) identifying the second protein from the cDNA of the protein-specific phagemid.

2. The method of claim 1, wherein DNA from the complex-forming antibody phage is amplified and used to increase the titer of the antibody phage(s) prior to the second screening step.

3. A method of identifying a first protein, or a second protein containing an idiotypic region of the first protein, from a tissue of interest by ribosome-phage screening, said use comprising:
(a) contacting said first protein with an antibody phagemid display library to form a complex between said protein and at least one member of said library;
(b) screening a ribosome display library of the proteome of the tissue of interest with the complex-forming antibody phage to identify a protein-specific ribosome that displays a second protein that binds the antibody phagemid; and
(c) identifying the second protein from the cDNA of the protein-specific ribosome.
